(19) United States
(12) Patent Application Publication
(10) Pub. No.: US 2005/0002894 A1
(43) Pub. Date: Jan. 6, 2005

(54) Novel uses of apple seed extracts in cosmetic or pharmaceutical compositions

(76) Inventors: Dirk Petersohn, Koeln (DE); Kordula Schlotmann, Duesseldorf (DE); Claudia Jassoy, Duesseldorf (DE); Marianne Waldmann-Laue, Monheim (DE); Sevda Yuecel, Neuss (DE)

Correspondence Address: HENKEL CORPORATION, THE TRIAD, SUITE 200, 2200 RENAISSANCE BLVD., GULPH MILLS, PA, 19406, US

(21) Appl. No.: 10/872,597

(22) Filed: Jun. 21, 2004

Related U.S. Application Data
(63) Continuation of application No. PCT/EP02/14122, filed on Dec. 12, 2002.

(30) Foreign Application Priority Data
Dec. 21, 2001 (DE) 101 63 246.0

(51) Int. Cl. A61K 7/06; A61K 35/78

(52) U.S. Cl. 424/74; 424/765

(57) ABSTRACT

Novel topical cosmetic and pharmaceutical compositions containing apple core extracts, and novel uses of apple core extracts in topical cosmetic or pharmaceutical compositions.

### CROSS REFERENCE TO RELATED APPLICATIONS

This application is a continuation under 35 U.S.C. § 365(c) and 35 U.S.C. §120 of international application PCT/EP02/14122, filed on Dec. 12, 2002. This application also claims priority under 35 U.S.C. § 119 of DE 101 63 246.0, filed Dec. 21, 2001, which is incorporated herein by reference in its entirety.

### BACKGROUND OF THE INVENTION

The present invention relates to novel topical cosmetic and pharmaceutical compositions which contain apple core extracts, and to novel uses of apple seed extracts in topical cosmetic or pharmaceutical compositions.

Cosmetic compositions which contain apple seed extracts are already known in the prior art. Japanese laid-open specification JP 11071294 A discloses the combination of apple seed and/or grape seed extracts with extracts of Saxifraga as a collagenase inhibitor in cosmetic compositions. The active ingredient combination prevents skin wrinkles and decreases these, it moreover eliminates active oxygen.

Japanese laid-open specification JP 53044639 A discloses cosmetic compositions containing apple seed extracts which stimulate the circulation, improve the care of the skin and its appearance and, on account of the absorption maximum at a wavelength of 270 nm, serve as UV protective agents. An antiinflammatory effect is moreover disclosed. Haircare preparations containing apple seed extract improve the sheen and the softness of the hair. Russian laid-open specification RU 2150263 discloses a night cream which contains an apple seed extract in combination with tocopheryl acetate and magnesium sulfate.

According to the invention, the generally used term “skin” is to be understood as meaning the skin itself, the mucous membrane and the skin appendages, if they include living cells, in particular the hair follicle, hair root, hair bulb, the ventral epithelium of the nail bed (lectulus), and sebaceous glands and sweat glands.

The human skin with its appendages is an organ of very complex construction, which consists of a large number of different cell types. Each living cell of this organ is able to react to signals from its environment, such as, for example, the action of topically applied cosmetics. These reactions of the cells are realized by an ordered regulation of gene expression, so that the metabolism of cells of the skin is not static, but very dynamic. The reactions of the skin to changes in the environment must not be considered, however, as reactions of individual, isolated cells. Rather, each cell is integrated into a complex communication network. This network comprises, for example, the communication between cells of the epidermis and cells of the dermis. Signal molecules, for example interleukins and growth factors, (e.g. KGF (keratino-cyte growth factor), EGF (epidermal growth factor) or FGF (fibroblast growth factor) are involved in the communication between the cells of the skin.

The investigation of active ingredient effects on individual, isolated cell types of the skin (e.g. fibroblasts, keratinocytes) is only able to yield an incomplete impression of the reactions actually occurring in the organ.

Tests of cosmetic active ingredients are as a rule carried out with the pure active ingredient on isolated cells of the skin (fibroblast or keratinocyte cultures). The investigation of a cosmetic active ingredient in combination with a galenic formulations is, however, very desirable, as the use situation of the cosmetic is simulated in this way. Interactions between the active ingredient and further components of the formulation cannot be excluded in advance. Such interactions are, however, only detectable if a galenic formulation is tested instead of an isolated active ingredient.

The application of the pure active ingredient to single-cell cultures of the skin is, in addition to the problems described above, also critical because of the skin barrier lacking in the single-cell culture. The question thus remains unanswered of whether the active ingredient is able anyway to penetrate the barrier of the skin from a galenic formulation and to reach the living cells of the organ, the actual site of action.

In the prior art, such investigations were, however, previously still not carried out routinely. Suitable skin model systems have in fact already been known for a relatively long time, but are scarcely utilized.

### SUMMARY OF THE INVENTION

The novel uses according to the invention of apple seed extracts in topical cosmetic or pharmaceutical compositions are based on effects which are surprising and are not to be predicted by the person skilled in the art, which have been determined in vitro on a human-homologous whole-skin model with subsequent investigations of the gene expression and the protein synthesis and have been demonstrated phenomenologically by means of histological sections and by means of the hyaluronic acid content.

The human-homologous skin model employed has a stratum corneum having a barrier function. Thus it was possible to determine effects of apple seed extracts in a manner close to reality by means of a galenic formulation. In this model, interactions between ingredients of the galenic formulation were taken into account just as interactions between various cells of the skin.

The action of an apple seed extract-containing cream formulation on the expression of 12 genes which are involved, inter alia, on cytoskeleton formation, the formation of communication pores between skin cells, cell adhesion to the extracellular matrix, lipid synthesis in the skin or generally in skin ageing, was determined with the aid of a DNA chip. Furthermore, the epidermal thickness of the skin model was measured, and its hyaluronic acid content and the RNA yield were determined. The identification of the genes investigated is carried out by means of the “Swiss-Prot” number. Swiss-Prot is a protein sequence databank which has been developed by the Swiss Institute for Bioinformatics (SIB) and by the European Bioinformatics Institute (EBI).

### DETAILED DESCRIPTION OF THE INVENTION

A first object of the present invention is topical cosmetic or pharmaceutical compositions which, in a suitable carrier, contain at least one apple seed extract and at least one active ingredient, selected from organic, inorganic and modified inorganic light protection filters, protein hydrolyzates and their derivatives, mono-, oligo- and polysaccharides, and their derivatives, α-hydroxycarboxylic acids and α-ketocarboxylic acids, and their ester, lactone or salt forms.

Within the meaning of the present invention, the cosmetic compositions according to the invention are used only for nontherapeutic treatment.

A further object of the present invention is the use of apple seed extracts for the production of topical cosmetic or pharmaceutical compositions for improving the mechanical stability of the skin, the skin appendages and the hair.

In the whole-skin model treated with apple seed extracts, it was possible to demonstrate an increase in the expression of the keratins 1, 5, 10 and 14 (KRT 1, KRT 5, KRT 10 and KRT 14), the “gap junction proteins” connexin 26 and connexin 43 and the hyaluronic acid receptor CD 44. The relationship between these proteins and the mechanical stability of the skin, the skin appendages and the hair is explained below.

The skin, as a surface of contact with the environment, is exposed to particular mechanical stresses. For the homeostasis of the skin it is therefore important to strengthen and to assist the skin in its mechanical protective function. The mechanical stressability of the skin is realized by different biological functionalities. Thus each cell, for example, is equipped with a “cytoskeleton”, which consists of filaments of different sizes. The cells of the epidermis contain, in particular, certain intermediary filaments, “keratins”, which are differentiated into keratins of type I and of type II. The keratins KRT 10 and KRT 14 belong to the type I keratins, the keratins KRT 1 and KRT 5 to the type II keratins. Depending on the differentiation state of the keratinocytes, the cells synthesize different keratins of types I and II, which in each case arrange together (dimerize) in the cell in pairs. In the basal keratinocytes, the keratins KRT 5 and KRT 14, which also serve as markers of dividing keratinocytes, are expressed and dimerize with one another. In the suprabasal keratinocytes, the keratins KRT 10 and KRT 1, which also serve as markers of differentiating keratinocytes, are expressed and likewise dimerize with one another. As a result of the increase in the expression of the keratins mentioned, the cytoskeleton is reinforced, which contributes to the mechanical stability. The simultaneously increased number and differentiation of the keratinocytes likewise contributes to the mechanical stability.

Cream formulations containing apple seed extracts increased the expression of the keratins KRT 5 and KRT 14 by the factors 2.3 and 2.4 respectively. The expression of the keratins KRT 10 and KRT 1 was increased by the factors 3.4 and 3.5 respectively by cream formulations containing apple seed extracts.

The increase in the keratinocyte number was confirmed by the increase in the total RNA yield demonstrated in the skin model and by the increase in the epidermal thickness likewise demonstrated. Skin models which had been treated with apple seed extract-containing cream formulations showed an increased total RNA yield after six and 48 hours' incubation. This indicates that apple seed extracts exert a general influence on the cell metabolism of the skin model and increase this. The term. “metabolism” is used in this connection in the sense of an increase in the proliferation and the protein synthesis, not in the sense of a metabolic metabolism.

A further object of the present invention is the use of apple seed extracts for the production of topical cosmetic or pharmaceutical compositions for improving the mechanical stability of the skin and the skin appendages, the mechanical stability being caused by the increase in the number of keratinocytes in the skin and the skin appendages.

A further object of the present invention is the use of apple seed extracts for the production of topical cosmetic or pharmaceutical compositions for improving the mechanical stability of the skin and the skin appendages, the mechanical stability being caused by the increase in the keratinocyte differentiation in the skin and the skin appendages.

The mechanical stability of the skin and of the skin appendages is furthermore assisted by the formation of “gap junctions”.

As already explained, the cells of the skin are not to be considered as individual isolated units. On the contrary, skin cells are integrated into a complex communications network. The “gap junctions” between two cells make up part of this network. These are structures in the cell membrane which serve for the formation of open channels to adjacent cells. Via these channels, low molecular weight signal substances can pass from one cell to the next and thus bring about communication. Certain proteins, the “connexins”, are involved in the formation of the “gap junctions”. The treatment of skin models with apple seed extracts resulted in an increased expression of the genes for connexin 43 and connexin 26, two proteins which also form, as is known, “gap junctions” in the skin and are thus involved in the formation of communication pores between skin cells. The “gap junctions”, however, do not only provide, for improved cell communication at the chemical level, but also increase the intercellular cell adhesion in the skin and the skin appendages.

A further object of the present invention is the use of apple seed extracts for the production of topical cosmetic or pharmaceutical compositions for improving the mechanical stability of the skin and the skin appendages, the mechanical stability being caused by the increase in the intercellular cell adhesion in the skin and the skin appendages.

In addition to the elements of the cytoskeleton and the gap junction proteins, further proteins impart mechanical stability to the skin tissue. These proteins bring about a binding between cells and extracellular matrix (ECM). A constituent of the ECM is, for example, collagen or alternatively hyaluronic acid. For the binding of cells to the proteins of the ECM, receptors are responsible which are located on the cell surface. Thus the surface receptor CD44, for example, whose expression is increased by apple seed extracts, binds to hyaluronic acid.

A further object of the present invention is the use of apple seed extracts for the production of topical cosmetic or pharmaceutical compositions for improving the mechanical stability of the skin and the skin appendages, the mechanical stability being caused by the increase in the adhesion between cells and the extracellular matrix in the skin and the skin appendages.

The use according to the invention of apple seed extracts for improving the mechanical stability is not restricted to the living cells of the skin and the skin appendages defined above, but also to the hair including the dead cells of the hair shaft. The site of formation of the hair is in the skin of the hair follicle, which consists of various specialized epithelial and connective tissue cells. Hair follicles pass through various growth phases, hair being produced in the anagenic phase. In the catagenic phase, however, the hair production decreases, the hair follicle atrophies, and finally hair production is stopped. The hair follicle is then in the resting state or in the “telogenic” phase. During the anagenic phase of the hair follicle, certain cell lines in the hair follicle synthesize epithelial keratins. The keratins 1 and 10, for example, also occur in the hair follicle on the “internal root sheath” (on the cuticle and the “Huxley's layer”). In addition, the cells of the “external root sheath” produce the keratins 5 and 14. Epidermal cells, that is likewise the keratinocytes of the “internal” and “external root sheath”, react to the treatment with apple seed extracts with the increased production of the keratins 1, 10, 5 and 14. It is to be assumed that an increased production of the hair-specific keratins occurs simultaneously. This reaction is accompanied by an increase in the keratinocyte number, an effect which is advantageous for the stability of the entire hair, that is also the dead hair shaft.

A further object of the present invention is the use of apple seed extracts for the production of topical cosmetic or pharmaceutical compositions for stimulating the endogenous barrier function of the skin and the skin appendages.

In the whole-skin model treated with apple seed extracts, it was possible to demonstrate an increase in the expression of the enzyme FAS (fatty acid synthase) and of the fatty acid binding protein (FABE). The relationship between these proteins and the endogenous barrier function of the skin and of the skin appendages is explained below. Epidermal keratinocytes produce and secrete lipids in order, for example, to maintain the barrier function of the skin. The composition of the lipids in the living cell layers of the epidermis is comparable with the lipid composition of other epithelia: phospholipids and cholesterol as the main constituent of cell membrane bilayers and triglycerides as energy carriers are the main lipids of the epidermis. In the stratum granulosum, a specific layer of the epidermis, lipids are packed very tightly into “granules”. During the terminal differentiation to the stratum corneum, the horny layer of the epidermis, these granules are secreted into the extracellular space, where they then form multiple lipid bilayers between the corneocytes.

During the terminal differentiation of the keratinocytes, their lipid composition changes drastically. Thus the phospholipid content decreases rapidly in the course of differentiation. The stratum corneum is finally almost free of phospholipids, while the proportion of ceramides, sterols and free fatty acids increases.

The enzyme FAS (fatty acid synthase), whose expression, as described above, is increased by apple seed extracts, is involved in the biosynthesis of free fatty acids. It is a relatively large protein, consisting of over 2500 amino acids, having a molecular weight of more than 273 kilodaltons (kDa). FAS catalyzes the production of long-chain free fatty acids from acetyl-CoA, malonyl-CoA and NADPH. Immunohistochemical investigations in the past have already shown that FAS is produced strongly in the stratum granulosum and moderately in the upper layers of the stratum spinosum.

In addition to the biosynthesis of the free fatty acids, their transport in the cell is likewise of biological importance. In the epidermis, the protein FABE (fatty acid binding protein in epidermis), inter alia, was described, which binds free fatty acids and appears to be involved in their transport. FABE, whose expression, as described above, is increased by apple seed extracts, preferably binds C18 fatty acids, the binding affinity becoming lower with increasing number of double bonds or a shortening of the chain length of the fatty acids.

For the chemical processes which are involved in the endogenous barrier function of the skin, cell communication by intercellular communication pores, the “gap junctions”, is indispensable.

A further object of the present invention is the use of apple seed extracts for the production of topical cosmetic or pharmaceutical compositions for stimulating the endogenous barrier function of the skin and the skin appendages, the stimulation being caused by an increase in the lipid production in the epidermis.

A further object of the present invention is the use of apple seed extracts for the production of topical cosmetic or pharmaceutical compositions for stimulating the endogenous barrier function of the skin and the skin appendages, the stimulation being caused by an increase in the formation of intercellular communication pores in the skin and the skin appendages and by the improvement of the intercellular cell communication.

A further object of the present invention is the use of apple seed extracts for the production of topical cosmetic or pharmaceutical compositions for increasing the expression of the keratin KRT5 having the Swiss-Prot number P13647, of the keratin KRT14 having the Swiss-Prot number P02533, of the keratin KRT10 having the Swiss-Prot number P13645, of the keratin KRT1 having the Swiss-Prot number P04264, of the gap junction protein beta 2 (connexin 26, CXB2) having the Swiss-Prot number P29033, of the gap junction protein alpha 1 (connexin 43) having the Swiss-Prot number P17302, of the hyaluronic acid receptor CD44 having the Swiss-Prot number P16070, of the fatty acid synthase (FAS.) having the enzyme classification EC 2.3.1.85 and the Swiss-Prot number P49327, of the epidermal fatty acid binding protein FABE having the Swiss-Prot number Q01469, of the protein PSMD2 having the Swiss-Prot number Q13200 and of the DNA binding protein A (DBPA) having the Swiss-Prot number P16989 in cells of the skin and the skin appendages.

A further object of the present invention is the use of apple seed extracts for the production of topical cosmetic or pharmaceutical compositions for inhibiting the expression of the bone proteoglycan II precursor (PGS2) having the Swiss-Prot number P07585 in cells of the skin and the skin appendages.

It was possible to demonstrate an inhibition of the expression of PGS2 (decorin) in the whole-skin model treated with apple seed extracts, which, as had been found in preliminary experiments, is expressed in increased extent in aged skin in comparison with young skin. The proteins CD44 (hyaluronic acid receptor), CXB2 (connexin 26), PSMD2 (Swiss-Prot number Q13200), DBPA (Swiss-Prot number P16989 and FAS (Swiss-Prot number P49327), which are expressed to a decreased extent in aged skin compared with young skin, were activated demonstrably in the whole-skin model by the treatment with apple seed extracts. Thus the topical treatment of the skin with apple seed extracts changes a gene expression profile, consisting of six age markers of the skin, in the direction of a gene expression profile of younger skin.

A particularly preferred apple seed extract according to the invention is the commercial product Ederline of the manufacturer Seporga. Ederline contains phyto-hormones, isoflavonoids, phytosterols, triterpenoids, tocopherols and natural waxes. In vitro tests with cultured skin cells show, according to the manufacturer's data, effects on the synthesis of collagen type I and collagen type III and also on fibronectin. In addition, in test subject studies a positive effect of Ederline on skin relief was measured. Moreover, the manufacturer's data make antioxidant and antiinflammatory actions valid.

The experimental investigations with Ederline have been carried out on isolated cells of the skin. It was not possible, however, to confirm described positive effects of Ederline on collagen type I and III and fibronectin in the whole-skin model. It is therefore presumed that the discoveries obtained on individual cells cannot be transferred here to the skin organ in its three-dimensional structure.

The product Ederline is firstly obtainable in water-soluble form as Ederline-H (INCI: PEG-40 Hydrogenated Castor Oil, PPG-2-Ceteareth-9, Pyrus Malus (Apple) Fruit Extract), on the other hand in fat-soluble form as Ederline-L (INCI: Hexyldecanol, Pyrus Malus (Apple) Fruit Extract).

Amounts of Ederline suitable according to the invention are 0.1-10% by weight, preferably 1-8% by weight and particularly preferably 3-5% by weight, in each case based on the total composition. Based on the content of active ingredients, apple seed extracts are employed according to the invention in amounts of from 0.001-2% by weight, preferably 0.01-1.6% by weight and particularly preferably 0.03-1% by weight, in each case based on the total composition.

One object of the present invention are topical cosmetic or pharmaceutical apple seed extract-containing compositions which contain at least one organic or inorganic or modified inorganic light protection filter. The light protection filters are substances present at room temperature in liquid or crystalline form, which are able to absorb ultraviolet rays and to give off the absorbed energy again in the form of longer-wavelength radiation, e.g. heat. A distinction is made between UVA filters and UVB filters. The UVA and UVB filters can be employed both individually and as mixtures. The use of filter mixtures is preferred according to the invention. The organic UV filters used according to the invention are selected from the derivatives of dibenzoylmethane, cinnamic acid esters, diphenylacrylic acid esters, benzophenone, camphor, p-aminobenzoic acid esters, o-aminobenzoic acid esters, salicylic acid esters, benzimidazoles, 1,3,5-triazines, monomeric and oligo-meric 4,4-diarylbutadienecarboxylic acid esters and carboxamides, ketotricyclo(5.2.1.0)decane, benzal-malonic acid esters, and any desired mixtures of the components mentioned. The organic UV filters can be oil-soluble or water-soluble. Particularly preferred oil-soluble UV filters according to the invention are 1-(4-tert-butylphenyl)-3-(4′-methoxyphenyl)propane-1,3-dione (Parsol® 1789), 1-phenyl)-3-(4′-isopropylphenyl)-propane-1,3-dione, 3-(4′-methylbenzylidene)-D,L-camphor, 2-ethylhexyl 4-(dimethylamino)benzoate, 2-octyl 4-(dimethylamino)benzoate, amyl 4-(dimethyl-amino)benzoate, 2-ethylhexyl 4-methoxycinnamate, propyl 4-methoxycinnamate, isopentyl 4-methoxycinnamate, 2-ethylhexyl 2-cyano-3,3-phenylcinnamate (octocrylene), 2-ethylhexyl salicylate, 4-isopropylbenzyl salicylate, homomenthyl salicylate (3,3,5-trimethylcyclohexyl salicylate), 2-hydroxy-4-methoxybenzophenone, 2-hydroxy-4-methoxy-4′-methylbenzophenone, 2,2′-di-hydroxy-4-methoxybenzophenone, di-2-ethylhexyl 4-methoxybenzmalonate, 2,4,6-trianilino-(p-carbo-2′-ethyl-1′-hexyloxy)-1,3,5-triazine (Octyl Triazone) and Dioctyl Butamido Triazone (Uvasorb® HEB), and any desired mixtures of the components mentioned.

Preferred water-soluble UV filters are 2-phenyl-benzimidazole-5-sulfonic acid and its alkali metal, alkaline earth metal, ammonium, alkylammonium, alkanol-ammonium and glucammonium salts, sulfonic acid derivatives of benzophenones, preferably 2-hydroxy-4-methoxybenzophenone-5-sulfonic acid and its salts, sulfonic acid derivatives of 3-benzylidenecamphor, such as, for example, 4-(2-oxo-3-bornylidenemethyl)benzene-sulfonic acid and 2-methyl-5-(2-oxo-3-bornylidene)-sulfonic acid and their salts.

The preferred inorganic light protection pigments according to the invention are finely dispersed metal oxides and metal salts, for example titanium dioxide, zinc oxide, iron oxide, aluminum oxide, cerium oxide, zirconium oxide, silicates (talc) and barium sulfate. The particles should in this case have a mean diameter of less than 100 nm, preferably between 5 and 50 nm and in particular between 15 and 30 nm, “nanopigments”. They can have a spherical form, but those particles can also be used which have an ellipsoidal shape or one differing from the spherical shape in another manner. The pigments can also be present in surface-treated, i.e. hydrophilized or hydrophobized, form. Typical examples are coated titanium dioxides, such as, for example, titanium dioxide T 805 (Degussa) or Eusolex® T2000 (Merck). Possible hydrophobic coating agents in this case are especially silicones and in this case especially trialkoxyoctylsilanes or simethicones. Titanium dioxide and zinc oxide are particularly preferred.

The light protection filters are present in the compositions according to the invention in amounts of from 0.1-30% by weight, preferably 1-20% by weight and particularly preferably 2-15% by weight, in each case based on the total composition.

Further objects of the present invention are topical cosmetic or pharmaceutical apple seed extract-containing compositions which contain at least one protein hydrolyzate or its derivative. According to the invention, both vegetable and animal protein hydrolyzates can be employed. Animal protein hydrolyzates are, for example, elastin, collagen, keratin, silk and lactoprotein hydrolyzates, which can also be present in the form of salts. Vegetable protein hydrolyzates, e.g. soybean, wheat, almond, pea, potato and rice protein hydrolyzates, are preferred according to the invention. Appropriate commercial products are, for example, DiaMin® (Diamalt), Gluadin® (Cognis), Lexein® (Inolex) and Crotein® (Croda). Even though the use of the additional protein hydrolyzates as such is preferred, it is optionally also possible to employ in their place amino acid mixtures obtained in another way or individual amino acids such as; for example, arginine, lysine, histidine or pyroglutamic acid. The use of derivatives of the protein hydrolyzates, e.g. in the form of their fatty acid condensation products, is likewise possible. Appropriate commercial products are, for example, Lamepon® (Cognis), Gluadin® (Cognis), Lexein® (Inolex), Crolastin® or Crotein® (Croda).

Also employable according to the invention are cationized protein hydrolyzates, where the underlying protein hydrolyzate can originate from animals, from plants, from marine life forms or from protein hydrolyzates obtained biotechnologically. Cationic protein hydrolyzates are preferred whose protein content has a molecular weight from 100 up to 25,000 daltons, preferably 250 to 5000 daltons. Furthermore, cationic protein hydrolyzates are to be understood as meaning quaternized amino acids and their mixtures. The cationic protein hydrolyzates can also be further derivatized. As typical examples of cationic protein hydrolyzates and derivatives used according to the invention, some of the products commercially obtainable may be mentioned under their INCI names: Cocodimonium Hydroxypropyl Hydrolyzed Collagen, Cocodimonium Hydroxypropyl Hydrolyzed Casein, Steardimonium Hydroxy propyl Hydrolyzed Collagen, Steardimonium Hydroxy-propyl Hydrolyzed Collagen, Steardimonium Hydroxypropyl Hydrolyzed Hair Keratin, Lauryldimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat protein, Cocodimonium Hydroxypropyl Silk Amino Acids, Hydroxypropyl Arginine Lauryl/Myristyl Ether HCl, Hydroxypropyltrimonium Gelatin. The plant-based cationic protein hydrolyzates and derivatives are very particularly preferred.

In the compositions according to the invention, the protein hydrolyzates and their derivatives are present in amounts of from 0.01-10% by weight, preferably 0.1-5% by weight and particularly preferably 0.1-3% by weight, in each case based on the total composition.

An additional object of the present invention are topical cosmetic or pharmaceutical apple seed extract-containing compositions which furthermore contain at least one mono-, oligo- or polysaccharide or their derivatives, saccharides which contain aminosugar units being excluded.

Monosaccharides suitable according to the invention are, for example, glucose, fructose, galactose, arabinose, ribose, xylose, lyxose, allose, altrose, mannose, gulose, idose and talose, and the deoxy sugars fucose and rhamnose. Glucose, fructose, galactose, arabinose and fucose are preferred; glucose is particularly preferred.

Oligosaccharides suitable according to the invention are composed of two to ten monosaccharide units, e.g. sucrose, lactose or trehalose. A particularly preferred oligosaccharide is sucrose. The use of honey, which mainly contains glucose and sucrose, is likewise particularly preferred.

Suitable polysaccharides according to the invention are composed of more than ten monosaccharide units. Preferred polysaccharides are the starches constructed of α-D-glucose units, and starch degradation products such as amylose, amylopectin and dextrins. According to the invention, chemically and/or heat-modified starches, e.g. hydroxypropylstarch phosphate, dihydroxypropyldistarch phosphate or the commercial products Dry Flo® are particularly advantageous. Dextrans and their derivatives, e.g. dextran sulfate, are furthermore preferred. Nonionic cellulose derivatives, such as methylcellulose, hydroxypropyl-cellulose, hydroxypropylmethylcellulose or hydroxy-ethylcellulose, and cationic cellulose derivatives, e.g. the commercial products Celquat® and Polymer JR®, and preferably Celquat® H 100, Celquat® L 200 and Polymer JR® 400 (polyquaternium-10), and polyquaternium-24 are likewise preferred. Further preferred examples are polysaccharides consisting of fucose units, e.g. the commercial product Fucogel®.

In the compositions according to the invention, the mono-, oligo- or polysaccharides or their derivatives are present in amounts of from 0.1-10% by weight, preferably 0.5-5% by weight and particularly preferably 1.0-3% by weight, in each case based on the total composition.

A further object of the present invention are topical cosmetic or pharmaceutical apple seed extract-containing compositions which furthermore contain at least one α-hydroxycarboxylic acid or α-ketocarboxylic acid or its ester, lactone or salt form. Suitable α-hydroxycarboxylic acids or α-ketocarboxylic acids are selected from lactic acid, tartaric acid, citric acid, 2-hydroxybutanoic acid, 2,3-dihydroxypropanoic acid, 2-hydroxypentanoic acid, 2-hydroxyhexanoic acid, 2-hydroxyheptanoic acid, 2-hydroxyoctanoic acid, 2-hydroxydecanoic acid, 2-hydroxydodecanoic acid, 2-hydroxytetradecanoic acid, 2-hydroxyhexadecanoic acid, 2-hydroxyoctadecanoic acid, mandelic acid, 4-hydroxymandelic acid, malic acid, erythraric acid, threaric acid, glucaric acid, galactaric acid, mannaric acid, gularic acid, 2-hydroxy-2-methylsuccinic acid, gluconic acid, pyruvic acid, glucuronic acid and galacturonic acid. The esters of the acids mentioned are selected from the methyl, ethyl, propyl, isopropyl, butyl, amyl, pentyl, hexyl, 2-ethylhexyl, octyl, decyl, dodecyl and hexadecyl esters. The α-hydroxycarboxylic acids or α-ketocarboxylic acids or their derivatives are present in amounts of from 0.1-10% by weight, preferably 0.5-5% by weight, in each case based on the total composition.

Advantageously, the compositions according to the invention can contain at least one synthetic film-forming, emulsion-stabilizing, thickening or adhesive polymer, selected from polymers which can be cationically, anionically or amphoterically charged or nonionic.

According to the invention, cationic, anionic and nonionic polymers are preferred.

Among the cationic polymers, polysiloxanes having quaternary groups, e.g. the commercial products Q2-7224 (Dow Corning), Dow Corning® 929 emulsion (with Amodimethicone), SM-2059 (General Electric), SLM-55067 (Wacker) and Abil®-Quat 3270 and 3272 (Th. Goldschmidt) are preferred.

Preferred anionic polymers contain carboxylate and/or sulfonate groups and, as monomers, for example acrylic acid, methacrylic acid, crotonic acid, maleic anhydride and 2-acrylamido-2-methylpropanesulfonic acid. In this case, the acidic groups can be present completely or partly as the sodium, potassium, ammonium, mono- or triethanolammonium salt. Preferred monomers are 2-acrylamido-2-methylpropanesulfonic acid and acrylic acid. As the sole monomer or as a comonomer, very particularly preferred anionic polymers contain 2-acrylamido-2-methylpropanesulfonic acid, where the sulfonic acid group can be present completely or partially in salt form. Within this embodiment, it is preferred to employ copolymers of at least one anionic monomer and at least one nonionic monomer. With respect to the anionic monomers, reference is made to the abovementioned substances. Preferred nonionic monomers are acrylamide, methacrylamide, acrylic acid esters, methacrylic acid esters, vinylpyrrolidone, vinyl ethers and vinyl esters. Preferred anionic copolymers are acrylic acid-acrylamide copolymers and in particular polyacrylamide copolymers with monomers containing sulfonic acid groups. A particularly preferred anionic copolymer consists of 70 to 55 mol % of acrylamide and 30 to 45 mol % of 2-acrylamido-2-methylpropanesulfonic acid, the sulfonic acid groups being completely or partially present as the sodium, potassium, ammonium, mono- or triethanolammonium salt. This copolymer can also be present in crosslinked form, the crosslinking agents employed preferably being polyolefinically unsaturated compounds such as tetraallyloxyethane, allylsucrose, allylpentaerythritol and methylene-bisacrylamide. Such a polymer is present in the commercial product Sepigel®305 from SEPPIC. The use of this compound has proven particularly advantageous within the context of the teaching according to the invention. The sodium acryloyldimethyltaurate copolymers with acrylamide or hydroxyethyl acrylate or sodium acrylate marketed under the names Simulgel® 600, Simulgel®NS and Simulgel®EG as a compound with isohexadecane or squalane and polysorbate-80 or polysorbate-60 have also proven particularly effective according to the invention.

Further particularly preferred anionic homo- and copolymers are uncrosslinked and crosslinked poly-acrylic, acids. In this case, allyl ethers of penta-erythritol, of sucrose and of propylene can be preferred crosslinking agents. Such compounds are, for example, the commercial products Carbopol®. As a monomer, a particularly preferred anionic copolymer contains an unsaturated, if desired substituted C₃₋₆-carboxylic acid or its anhydride to 80-98% and, if desired, substituted acrylic acid esters of saturated C₁₀₋₃₀-carboxylic acids to 2-20%, where the copolymer can be crosslinked using the abovementioned crosslinking agents. Appropriate commercial products are Pemulen® and the Carbopol® types 954, 980, 1342 and ETD 2020 (ex B. F. Goodrich).

Suitable nonionic polymers are, for example, polyvinyl alcohols, which can be partially hydrolyzed, e.g. the commercial products Mowiole, and vinyl pyrrolidone/vinyl ester copolymers and polyvinylpyrrolidones, which are marketed, for example, under the trade name Luviskol® (BASF).

Advantageously, the skin treatment compositions according, to the invention are present in the form of a liquid or solid oil-in-water emulsion, water-in-oil emulsion, multiple emulsion, microemulsion, PIT emulsion or Pickering emulsion, nanoemulsion, of a hydrogel, of a lipogel, of a single- or multiphase solution, of a foam, of a powder or of a mixture with at least one polymer suitable as a medical adhesive. The compositions can also be administered in anhydrous form, such as, for example, an oil or a balsam. In this context, the carrier can be a vegetable or animal oil, a mineral oil, a synthetic oil or a mixture of such oils.

In a particular embodiment of the compositions according to the invention, the compositions are present as a microemulsion. In addition to the thermo-dynamically stable microemulsions, microemulsions in the context of the invention are also understood as meaning the “PIT” emulsions. These emulsions are systems containing the 3 components water, oil and emulsifier, which are present at room temperature as an oil-in-water emulsion. On warming these systems, micro-emulsions are formed in a certain temperature range (designated as the phase inversion temperature or “PIT”), which are converted to water-in-oil emulsions on further warming. On subsequent cooling, O/W emulsions are again formed, which, however, are present even at room temperature as microemulsions or as very finely divided emulsions having an average particle diameter of below 400 nm and in particular of approximately 100-300 nm.

According to the invention, such micro- or “PIT” emulsions can be preferred which have an average particle diameter of approximately 200 nm.

In the embodiment as an emulsion or as a surfactant solution, e.g. as cleansing agents, the compositions according to the invention contain at least one. surface-active substance as an emulsifier or dispersing agent. Suitable emulsifiers are, for example, addition products of 4 to 30 mol of ethylene oxide and/or 0 to 5 mol of propylene oxide to linear C₈-C₂₂-fatty alcohols, to C₁₂-C₂₂-fatty acids and to C₈-C₁₅-alkylphenols, C₁₂-C₂₂-fatty acid mono- and diesters of addition products of 1 to 30 mol of ethylene oxide to C₃-C₆-polyols, in particular to glycerol, ethylene oxide and poly-glycerol addition products to methyl glucoside fatty acid esters, fatty acid alkanolamides and fatty acid glucamides, C₈-C₂₂-alkyl mono- and oligoglycosides and their ethoxylated analogs, degrees of oligomerization of 1.1 to 5, in particular 1.2 to 2.0, and glucose as the sugar component being preferred, mixtures of alkyl (oligo)glucosides and fatty alcohols, e.g. the commercially obtainable product Montanov®68, addition products of 5 to 60 mol of ethylene oxide to castor oil and hardened castor oil, partial esters of polyols having 3-6 carbon atoms with saturated. C₈-C₂₂-fatty acids, sterols, in particular cholesterol, lanosterol, beta-sitosterol, stigmasterol, campesterol and ergosterol, and mycosterols, phospholipids, especially glucose phospholipids, fatty acid esters of sugars and sugar alcohols such as sorbitol, polyglycerols and polyglycerol derivatives, preferably polyglyceryl 2-di-polyhydroxystearate (commercial product Dehymuls®PGPH) and polyglyceryl 3-diisostearate (commercial product Lameform® TGI), and linear and branched C₈-C₃₀-fatty acids and their Na, K, ammonium, Ca, Mg and Zn salts.

The compositions according to the invention contain the emulsifiers preferably in amounts of from 0.1 to 25% by weight, in particular 0.5-15% by weight, based on the total composition.

In a particularly preferred embodiment, at least one nonionic emulsifier having an HLB of 8 and below is present. Suitable emulsifiers of this type are, for example, compounds of the general formula R¹—O—R², in which R¹ is a primary linear alkyl, alkenyl or acyl group having 20-30 C atoms and R² is hydrogen, a group of the formula —(CₙH₂ₙO)ₓ—H where x=1 or 2 and n=2-4 or a polyhydroxyalkyl group having 4-6 C atoms and 2-5 hydroxyl groups. Further preferably suitable emulsifiers having an HLB of 8 and below are the addition products of 1 or 2 mol of ethylene oxide or propylene oxide to behenyl alcohol, erucyl alcohol, arachidyl alcohol or alternatively to behenic acid or erucic acid. Preferably, the monoesters of C₁₆-C₃₀-fatty acids with polyols such as, for example, penta-erythritol, trimethylolpropane, diglycerol, sorbitol, glucose or methylglucose are also suitable. Examples of such products are sorbitan monobehenate or pentaerythritol monoerucate.

In another, likewise particularly preferred embodiment, at least one ionic emulsifier, selected from anionic, zwitterionic, ampholytic and cationic emulsifiers, is present. Preferred anionic emulsifiers are alkyl-sulfates, alkyl polyglycol ether sulfates and ether-carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates, and protein-fatty acid condensates. Particularly suitable zwitterionic emulsifiers are the “betaines”, such as the N-alkyl-N,N-dimethylammonium glycinates, N-acylaminopropyl-N,N-dimethylammonium glycinates and 2-alkyl-3-carboxymethyl-3-hydroxyethyl-imidazolines in each case having 8 to 18 C atoms in the alkyl or acyl group, and coconut acylaminoethyl-hydroxyethylcarboxymethyl glycinate. Examples of suitable ampholytic emulsifiers are N-alkylglycines, N-alkylaminopropionic acids, N-alkylaminobutyric acids, N-alkyliminodipropionic acids, N-hydroxyethyl-N-alkyl-amidopropylglycines, N-alkyltaurines, N-alkyl-sarcosines, 2-alkylaminopropionic acids and alkylamino-acetic acids in each case having approximately 8 to 24 C atoms in the alkyl group. The ionic emulsifiers are present in an amount of from 0.1 to 20% by weight, preferably from 1 to 15% by weight and particularly preferably from 2 to 10% by weight, based on the total composition.

Further suitable additives are fatty substances, in particular vegetable oils, such as sunflower oil, olive oil, soybean oil, rapeseed oil, almond oil, jojoba oil, orange oil, wheatgerm oil, peach kernel oil and the liquid components of coconut oil, liquid paraffin oils, isoparaffin oils and synthetic hydrocarbons, di-n-alkyl ethers having a total of 12 to 36 C atoms, e.g. di-n-octyl ether and n-hexyl n-octyl ether, fatty acids, particularly linear and/or branched, saturated and/or unsaturated C₈₋₃₀-fatty acids, fatty alcohols, particularly saturated, mono- or polyunsaturated, branched or unbranched fatty alcohols having 6-30 carbon atoms, ester oils, that is esters of C₆₋₃₀-fatty acids with C₂₋₃₀-fatty alcohols, alkyl hydroxy-carboxylates, the full esters of glycolic acid, lactic acid, malic acid, tartaric acid or citric acid being preferred, dicarboxylic acid esters such as di-n-butyl adipate, and diol esters such as ethylene glycol di-oleate or propylene glycol di(2-ethyl hexanoate), symmetrical, unsymmetrical or cyclic esters of carbonic acid with fatty alcohols, e.g. glycerol carbonate or dicaprylyl carbonate (Cetiolo CC), mono-, di- and trifatty acid esters of saturated and/or unsaturated linear and/or branched fatty acids with glycerol waxes, in particular insect waxes, plant waxes, fruit waxes, ozocerite, microwaxes, ceresin, paraffin waxes, triglycerides of saturated and optionally hydroxylated C₁₆₋₃₀-fatty acids, e.g. hardened triglyceride fats, silicone compounds, selected from decamethylcyclopenta-siloxane, dodecamethylcyclohexasiloxane and silicone polymers, which, if desired, can be crosslinked, e.g. polydialkylsiloxanes, polyalkylarylsiloxanes, ethoxylated polydialkylsiloxanes, and polydialkylsiloxanes which contain amine and/or hydroxy groups.

The amount of the fatty substances employed is 0.1-50% by weight, preferably 0.1-20% by weight and particularly preferably 0.1-15% by weight, in each case based on the total composition.

The compositions according to the invention can contain further active ingredients, excipients and additives, for example vitamins, provitamins and vitamin precursors from the groups A, B, C, E and F, allantoin, bisabolol, antioxidants, for example imidazoles (e.g. urocaninic acid) and their derivatives, chlorogenic acid and its derivatives, lipoic acid and its derivatives (e.g. dihydrolipoic acid), aurothio-glucose, propylthiouracil and other thiols (e.g. thio-redoxin, glutathione, cysteine, cystine, cystamine and their glycosyl, N-acetyl, methyl, ethyl, amyl, propyl, butyl, lauryl, palmitoyl, oleyl, γ-linoleyl, cholesteryl and glyceryl esters), and their salts, di-lauryl thiodipropionate, distearyl thiodipropionate, thiodipropionic acid and its derivatives (esters, ethers, peptides, lipids, nucleotides, nucleosides and salts), and sulfoximine compounds (e.g. buthionine sulfoximines, homocysteine sulfoximine, butionine sulfones, penta-, hexa- and heptathionine sulfoximine) in very low tolerable doses (e.g. pmol to μmol/kg), furthermore (metal) chelators (e.g. phytic acid, lactoferrin), humic acids, bile acids, bile extracts, bilirubin, biliverdin, ubiquinone and ubiquinol and their derivatives, coniferyl benzoate of benzoin resin, rutic acid and its derivatives, α-glycosylrutin, ferulic acid, furfurylideneglucitol, carnosine, butyl-hydroxytoluene, butylhydroxyanisole, nordihydro-guaiaretic acid, nordihydroguaiaretic acid, trihydroxy-butyrophenone, uric acid and its derivatives, catalase, superoxide dismutase, zinc and its derivatives (e.g. ZnO, ZnSO₄), selenium and its derivatives (e.g. selenomethionine), stilbenes and their derivatives (e.g. stilbene oxide, trans-stilbene oxide) and the derivatives suitable as antioxidants (salts, esters, ethers, sugars, nucleotides, nucleosides, peptides and lipids) of these active ingredients, furthermore ceramides and pseudoceramides, triterpenes, in particular triterpene acids such as ursolic acid, rosmaric acid, betulinic acid, boswellic acid and brionolic acid, monomeric catechols, particularly catechol and epicatechol, leukoanthocyanidines, catechol polymers (catechol tanning agents), and gallotannins, thickening agents, e.g. natural and synthetic clays and layer silicates such as bentonite, hectorite, montmorillonite or Laponite®, dimethyl isosorbide, alpha-, beta- and gamma-cyclodextrins, solvents, swelling agents and penetrants such as ethanol, isopropanol, ethylene glycol, propylene glycol, propylene glycol monoethyl ether, glycerol and diethylene glycol, carbonates, hydrogencarbonates, guanidines, ureas, and primary, secondary and tertiary phosphates, perfume oils, pigments and dyes for coloring the composition, substances for adjusting the pH, complexing agents such as EDTA, NTA, β-alaninedi-acetic acid and phosphonic acids, pearl luster agents such as ethylene glycol mono- and distearate, opacifying agents such as latex, styrene/PVP and styrene/acrylamide copolymers and propellants such as propane-butane mixtures, N₂O, dimethyl ether, CO₂ and air.

The following examples are intended to illustrate the present invention without restricting it hereto.

Experimental Section

1. Investigations on Multilayer Skin Models

The action of Ederline L was investigated on a multilayer in-vitro skin model. The skin model is a human skin equivalent, which consists of a dermis with fibroblasts and an epidermis of keratinocytes.

This multilayer structure is formed in a special culturing process. First, dermal equivalents (DE) were produced by pipetting a suspension of 2×10⁵/cm² fibroblasts of human foreskin in a culture medium onto a matrix consisting of chitosan, collagen and glycosaminoglycans (matrix described in Collombel, C. et al.: Biomaterials with a base of collagen, chitosans and glycosaminoglycans, process for preparing them and their application in human medicine, U.S. Pat. No. 5,166,187). The culture medium consisted of Dulbecco's Modified Eagle's Medium (DMEM), supplemented with 10% fetal calf serum (FCS), 25 μg/ml of gentamicin, 100 Ul/ml of penicillin, 1 μg/ml of amphotericin B, 50 μg/ml of sodium ascorbate and 4 mM L-glutamine. The dermal equivalents were incubated in this medium at 37° C. in an atmosphere of CO₂/air (5%/95%, v/v) and 90% atmospheric humidity for 14 days, the medium being replaced every day. For the skin equivalents (SE), keratinocytes from human foreskin were inoculated in a density of 200,000 cells/cm² onto the 14 day-old DEs and incubated under submerse conditions in a medium consisting of 60% DMEM, 30% HAM F12 and 10% FCS, supplemented with 25 μg/ml of gentamicin, 100 Ul/ml of penicillin, 1 μg/ml of amphotericin B, 50 μg/ml of sodium ascorbate, 4 mM L-glutamine, 10 ng/ml of epidermal growth factor (EGF), 0.4 μg/ml of hydrocortisone, 0.12 Ul/ml of insulin, 10⁻⁹ M cholera toxin, 5 ng/ml of transferrin and 180 μM adenine for a further 7 days. The skin equivalents were then cultured on the air-liquid interface for a further 14 days in modified keratinocyte medium (DMEM-HAM F12, supplemented with 0.4 μg/ml of hydrocortisone and 0.12 Ul/ml of insulin).

In comparison with monolayer cultures customarily used, this whole-skin model corresponds very much better to the in-vivo situation, since keratinocytes and fibroblasts are in close contact with one another and, as in vivo, can exchange signal substances.

2. Treatment of the Whole Skin Models:

For carrying out the tests, cream formulations based on the experimental recipe listed below were prepared with 3% by weight and with 5% by weight of Ederline L, the difference to 100% by weight being compensated with water. Subsequently, the effects of the apple seed extract-containing creams on whole skin models were assessed in comparison with a placebo cream (identical cream formulation without Ederline L). For this, the skin models were treated topically with 5 μl of the various cream formulations, subsequently incubated for 6 hours or 48 hours and the gene expression of the skin models treated with cream was determined in comparison with the gene expression of untreated skin models.

In a second test batch, whole-skin models were treated four times with the cream formulations mentioned over 9 days in total and effects at the histological level and the protein level were determined.

Cream Formulation of the Experimental Recipes (Data in % by Weight)

| | |
|---|---|
| Montanov ® 68 | 6.00 |
| Myritol ® 318 | 7.00 |
| Stenol ® 16/18 | 1.25 |
| Cutina ® MDV | 2.50 |
| Novata ® AB | 3.00 |
| Cetiol ® SB 45 | 1.50 |
| Eusolex ® 4360 | 0.50 |
| Tocopheryl acetate | 0.50 |
| Baysilon ® M350 | 0.50 |
| PHB propyl ester | 0.20 |
| Generol ® R | 0.50 |
| TiO₂ | 0.30 |
| Tego carbomer 2% strength | 10.00 |
| Talc Ger. Ph. | 0.50 |
| Glycerol | 4.50 |
| Sorbitol 70% strength | 2.00 |
| Methyl p-hydroxybenzoate | 0.20 |
| NaOH 10% strength | pH 4.8-5.2 |
| Water, dist. | to 100 |

3. RNA Preparation from the Whole-skin Models:

The RNA preparations were carried out 6 hours and 48 hours after the application of cream.

Before the isolation of the RNA (ribonucleic acid), the skin models were detached from the filter paper under ribonuclease-free conditions, frozen in a vessel using liquid nitrogen and subsequently stored in liquid nitrogen until work-up.

For RNA preparation, the models were worked up according to a modified protocol of Qiagen (RNeasy protocol: Isolation of total RNA from Heart, Muscle and Skin Tissue; TS-RY7; 05/99). The RNA was measured photometrically. The RNA yield (see table 1) already gives indications of the activity of a substance.

TABLE 1
RNA total yield of treated and untreated whole-skin
models after 6 hours and 48 hours [in μg of RNA]

| | | | treated with | treated with |
|---|---|---|---|---|
| | | | cream | cream |
| | | treated with | containing 3% | containing 5% |
| | | placebo | by weight of | by weight of |
| | Untreated | cream | Ederline L | Ederline L |
| After 6 | 34 | 27 | 42 | 79 |
| hours | | | | |
| After | 32 | 24 | 47 | 47 |
| 48 hours | | | | |

Compared with the untreated or with placebo cream-treated whole skin models, a marked increase in the RNA yield was seen on treatment with apple seed extract-containing creams.

4. Gene Expression Analyses

The gene expression analyses were carried out by means of cDNA arrays (arrays with complementary DNA). For the present study, cDNA arrays were used which carry different cDNAs of the species man and are all active in human skin. As a positive control, “housekeeping” genes and E. coli DNA fragments were additionally applied. As negative controls, herring sperm DNA and buffer were also applied. The PCR amplificates of the cloned cDNA fragments were adjusted to a uniform concentration and applied drop-wise to the surface of derivatized slides using a dispensing apparatus. For the determination of the gene expression profiles, RNA of a treated skin model was in each case labeled with the fluorescent dye Cy5 and the RNA of the corresponding untreated skin model (control) with the fluorescent dye Cy3 and jointly hybridized on a cDNA array. The labeling was achieved by reverse transcription with incorporation of fluorescence-labeled nucleotides (Cy3-dCTP or Cy5-dCTP). The hybridized arrays were read off using a laser scanning apparatus. The images obtained in each case for both fluorescence labelings are digitally overlaid for the continuing analysis. In this case, the color green means that the Cy5 fluorescence has a higher intensity than Cy3, red that the Cy3 fluorescence has a higher intensity than Cy5, yellow that both fluorescences have the same intensity and thus also the corresponding gene in both samples was expressed equally strongly.

For setting up the expression profiles, the Cy3 and Cy5 signal and background intensities of the hybridized arrays were firstly determined. The background values were subtracted from the signal intensities, the average mean value of the double spots was calculated and finally the quotient of Cy5/Cy3 signal was calculated. The values were standardized over the median of all signal quotients.

For the further evaluation, only those pairs of signals (Cy3 and Cy5) were used in which, after background subtraction, at least one of the two signal intensities was at least 3 times above the signal intensity of the negative controls (herring sperm DNA and buffer). It was thereby guaranteed that very weak signal intensities, which react extremely sensitively to a slightly varying background or a nonspecific hybridization, were excluded. For the assessment of the apple seed extract as an active ingredient, it was of interest to find out which genes in the whole-skin model are regulated by addition of the apple seed extract. In this case, effects which are based on an at least two-fold differential expression were significant and were assessed further.

TABLE 2
Relative gene expression in skin models
treated with 5% by weight Ederline L-containing cream
after 6 hours and after 48 hours, relative to untreated
skin and compared with the gene expression in aged skin
(69 years) in relation to young skin (29 years)

| | | Expression on | Expression on |
|---|---|---|---|
| | Expression | cream treatment | cream treatment |
| | in aged skin | with 5% by weight | with 5% by weight |
| | relative to | Ederline L after | Ederline L after |
| | expression | 6 h relative to | 48 h relative to |
| | in young | expression in | expression in |
| | skin | untreated skin | untreated skin |
| PGS 2 | +1.5 | −1.7 | −2.1 |
| CD 44 | −2.1 | +2.2 | +2.4 |
| GJB 2 | −2.1 | +2.9 | +2.1 |
| PSMD 2 | −2.1 | +2.5 | +1.6 |
| DBPA | −3.3 | +2.2 | +2.2 |
| FAS | −3.3 | +3.0 | +2.3 |

It can be inferred, for example, from table 2 that in aged skin (69 years) in relation to young skin (29 years) the gene PGS 2 is expressed more strongly by a factor of 1.5. The other genes listed are expressed more weakly in the aged skin by a factor of 2.1 or 3.3. The gene expression in skin models treated with apple seed extract cream compared with the gene expression in untreated skin models shows an opposite trend for each gene investigated. Thus the treatment with apple seed extracts modifies a gene expression profile, consisting of six age markers of the skin, in the direction of a gene expression profile of younger skin.

TABLE 3
Activation of the genes for connexin 43
and connexin 26 by treatment with apple seed extract in
relation to the placebo-treated skin model

| | | Expression | Expression |
|---|---|---|---|
| | | on cream | on cream |
| | | treatment | treatment |
| | | with 5% by | with 5% by |
| | Expression on | weight | weight |
| | placebo | Ederline L | Ederline L |
| | treatment | after 6 h | after 48 h |
| | relative to | relative to | relative to |
| | expression in | expression in | expression in |
| | untreated | untreated | untreated |
| | skin | skin | skin |
| Connexin 43 | 1.5 | 2.6 | 2.3 |
| Connexin 26 | 1.5 | 2.9 | 2.1 |

The treatment of skin models with apple seed extracts resulted in an increased expression of the genes for connexin 43 and connexin 26. The values above 2 are statistically significant, since they additionally take into account the biological variation.

TABLE 4
Activation of the genes for the proteins
keratin 10, keratin 1, keratin 5, keratin 14 and the
hyaluronate receptor CD 44 by treatment with apple seed
extract in relation to the placebo-treated skin model

| | | | Expression |
|---|---|---|---|
| | Expression | Expression on | on cream |
| | on placebo | cream treatment | treatment |
| | treatment | with 5% by | with 5% by |
| | relative to | weight Ederline | weight Ederline |
| | expression | L after 6 h | L after 48 h |
| | in | relative to | relative to |
| | untreated | expression in | expression in |
| | skin | untreated skin | untreated skin |
| KRT 10 | 1.3 | 3.4 | 2.0 |
| KRT 1 | 1.0 | 3.5 | 2.0 |
| KRT 5 | 1.1 | 2.3 | 1.5 |
| KRT 14 | 1.0 | 2.4 | 1.3 |
| CD 44 | 1.5 | 2.2 | 2.4 |

The treatment of skin models with apple seed extracts resulted in an increased expression of the genes for various keratins and the haluronate surface receptor CD 44. Thus the CD 44-expressing cells have an increased capacity to bind to extracellular hyaluronic acid. The values above 2 are statistically significant since they additionally take into account the biological variation.

TABLE 5
Activation of the genes for fatty acid synthase (FAS) and fatty
acid binding protein in epidermis (FABE) by treatment with
apple seed extract in relation to the placebo-treated skin model

| | | Expression on cream | Expression on |
|---|---|---|---|
| | Expression on | treatment with 5% | cream treatment |
| | placebo treatment | by weight Ederline | with 5% by weight |
| | after 48 h | L after 6 h | Ederline L after |
| | relative to | relative to | 48 h relative to |
| | expression in | expression in | expression in |
| | untreated skin | untreated skin | untreated skin |
| FAS | 1.4 | 3.0 | 2.3 |
| FABE | 1.5 | 2.4 | 1.7 |

The treatment of whole skin models with apple seed extract-containing creams led to an increased expression of the genes for FAS and FABE. It is to be assumed that this effect also acts at the level of the proteins and their activity, so that it can be assumed therefrom that the lipid content of the skin is raised by an increased lipid synthesis in the epidermis and thus the barrier function is improved.

5. Epidermal Thickness

The treatment time before measurement of the epidermal thickness was 9 days.

After the end of the treatment period, the skin models were embedded in OCT medium and sections having a thickness of 4 μm in each case were prepared by means of a cryostat.

For the control of the epidermal thickness, the preparations were stained (H & E staining) using hematoxylin/eosin, a staining technique customary in dermatological histology, and the layer thickness of the living cell layers (epidermis without stratum corneum) was measured on 3 different sections in each case per skin model (with n=3 parallel cultures) at 3 representative positions by means of image analysis.

After topical treatment for 9 days, the histological structure of the whole-skin model and the formation of the cell layers in all 3 types of treatment appeared normal. After treatment with apple seed extract-containing creams, the living layers of the epidermis were thickened in comparison with the placebo treatment. With increasing Ederline L concentration, an increase in the thickness of the stratum corneum was observed.

TABLE 6
Thickness of the living epidermal layers
(without stratum corneum) after topical treatment with
placebo cream, cream containing 3% by weight of Ederline L
and cream containing 5% by weight of Ederline L four times
over a total of 9 days.

| | | Cream | Cream |
|---|---|---|---|
| | | treatment | treatment |
| | | with 3% by | with 5% by |
| | Placebo | weight | weight |
| | treatment | Ederline L | Ederline L |
| Relative | 100 +/− 5% | 125 +/− 3% | 15 +/− 8 |
| layer | | | |
| thickness | | | |

The living layers of the epidermis are thickened in comparison with the placebo treatment, to be precise by about 25% on treatment with the cream containing 3% by weight of Ederline L and by about 15% on treatment with the cream containing 5% by weight of Ederline L.

6. Hyaluronic Acid Content

The hyaluronic acid content was determined from the medium with the aid of the “hyaluronic acid” kit (HA assay, Akagi Trading Co. Ltd., Kobe Hyogo, Japan) according to the manufacturer's instructions.

The whole-skin models were treated on experimental days 0, 2, 4 and 7 with the respective creams. The sampling for the analysis of the hyaluronic acid content was carried out on experimental days 1, 3 and 8, that is in each case one day after cream treatment had taken place. The treatment thus took place a total of four times within a period of 9 days.

TABLE 7
Hyaluronic acid content in the medium of
whole-skin models 1, 3 and 8 days after topical treatment
for the first time with placebo cream and cream containing
5% by weight of Ederline L [in μg of hyaluronic acid per
ml of medium].

| | | | treated |
|---|---|---|---|
| | | | with cream |
| | | treated | containing |
| | | with | 5% by |
| | | placebo | weight |
| | untreated | cream | Ederline L |
| Experimental | 818 | 1045 | 825 |
| day 1 | | | |
| Experimental | 1182 | 1318 | 1409 |
| day 3 | | | |
| Experimental | 1364 | 1591 | 1909 |
| day 8 | | | |

The treatment with the cream containing 5% by weight of Ederline L led to an increase in the hyaluronic acid synthesis, in particular after an 8-day treatment period.

TABLE 8
Composition of the genes investigated

| | Unigene | | |
|---|---|---|---|
| | Accession | Swiss Prot | |
| No. | No(s) | number | Description |
| 1 | Hs. 195850 | P13647 | KRT5: KERATIN, TYPE II CYTOSKELETAL 5 |
| | | | (CYTO-KERATIN 5) (K5) (CK 5) (58 KDA |
| | | | CYTOKERATIN) (epidermolysis bullosa |
| | | | simplex, Dowling-Meara/Kobner/Weber- |
| | | | Cockayne types) |
| 2 | Hs. 117729 | P02533 | Keratin 14 |
| 3 | Hs. 99936 | P13645 | KRT10: KERATIN, TYPE I CYTOSKELETAL 10 |
| | | | (CYTO-KERATIN 10) (K10) (epidermolytic |
| | | | hyperkeratosis; keratosis palmaris et |
| | | | plantaris) |
| 4 | Hs. 80828 | P04264 | Keratin 1 |
| 5 | Hs. 83190 | P49327 | FAS: FATTY ACID SYNTHASE (EC 2.3.1.85) |
| | | | [INCLUDES: EC 2.3.1.38; EC 2.3.1.39; EC |
| | | | 2.3.1.41; EC 1.1.1.100; EC 4.2.1.61; EC |
| | | | 1.3.1.10; EC 3.1.2.14]. |
| 6 | Hs. 153179 | Q01469 | FABE: FATTY ACID BINDING PROTEIN, |
| | | | EPIDERMAL (E FABP) (PSORIASIS |
| | | | ASSOCIATED FATTY ACID BINDING PROTEIN |
| | | | HOMOLOG) (PA FABP) (FABP5) |
| 7 | Hs. 1139 | P16989 | DBPA: HUMAN (CSDA OR DBPA) DNA BINDING |
| | | | PROTEIN A (COLD SHOCK DOMAIN |
| 8 | Hs. 74619 | Q13200 | PSMD2: (PSMD2 OR TRAP2) 26S PROTEASOME |
| | | | REGULATORY SUBUNIT S2 (P97) (TUMOR |
| | | | NECROSIS FACTOR TYPE 1 RECEPTOR |
| | | | ASSOCIATED PROTEIN 2). |
| 9 | Hs. 169610 | P16070 | CD44 |
| 10 | | P29033 | CXB2: GAP JUNCTION BETA 2 PROTEIN 26 kD |
| | | | (CONNEXIN 26) (CX26) |
| 11 | Hs. 74471 | P17302 | gap junction alpha 1 protein, 43 kD |
| | | | (connexin 43) |
| 12 | Hs. 76152 | P07585 | PGS2: BONE PROTEOGLYCAN II PRECURSOR |
| | | | (PG S2) (DECORIN) (PG40) |

### FURTHER RECIPE EXAMPLES

### Example 1

### Cream Formulation (Data in % by Weight)

| | |
|---|---|
| Isopropyl palmitate | 5.00 |
| Cutina ® MDV | 2.00 |
| Stenol ® 1618 | 1.00 |
| Baysilon ® M 350 | 0.50 |
| Biophilic ® H | 4.00 |
| 1,6-Hexanediol | 6.00 |
| Glycerol | 5.00 |
| Trilon ® A | 0.10 |
| Ederline ® L | 3.00 |
| Tego carbomer, 2% strength | 20.00 |
| Water | to 100 |

### Example 2

### Cream Formulation (Data in % by Weight)

| | |
|---|---|
| Emuliance ® | 4.00 |
| Myritol ® 318 | 6.00 |
| Cutina ® MDV | 2.00 |
| Stenol ® 1618 | 1.00 |
| Baysilon ® M 350 | 0.50 |
| 1,6-Hexanediol | 6.00 |
| Glycerol | 5.00 |
| Ederline L | 3.00 |
| Water | to 100 |

### Example 3

### Cream Formulation (Data in % by Weight)

| | |
|---|---|
| Montanov ® 202 | 3.00 |
| Isopropyl stearate | 3.00 |
| Myritol ® 331 | 1.00 |
| Performalene ® 400 | 1.00 |
| Cegesoft ® C 24 | 3.00 |
| Lanette ® 22 | 1.00 |
| Cutina ® MDV | 2.00 |
| Tocopheryl acetate | 0.50 |
| Controx ® KS | 0.25 |
| Parsol ® 1789 | 1.00 |
| Eusolex ® 6300 | 2.00 |
| Uvinul ® T 150 | 1.25 |
| Baysilon ® M350 | 0.50 |
| Tego carbomer 140 2% strength | 25.00 |
| 1,6-Hexanediol | 6.00 |
| Glycerol | 5.00 |
| 1,2-Propylene glycol | 5.00 |
| DSH-CN | 2.00 |
| NaOH 10% strength pH | 4.8-5.2 |
| Dry Flo Plus | 1.00 |
| Ederline L | 3.00 |
| Water demin. | to 100 |

### Example 4

### Cream Formulation (Data in % by Weight)

| | |
|---|---|
| Thistle oil | 3.0 |
| Myritol ® PC | 3.5 |
| Lanette ® 22 | 3.0 |
| Cutina ® GMS-V | 3.0 |
| Stenol ® 16/18 | 2.0 |
| Isopropyl stearate | 6.0 |
| Baysilon ® M350 | 1.0 |
| Controx ® KS | 0.05 |
| Propyl p-hydroxybenzoate | 0.2 |
| Glycerol | 5.0 |
| Methyl p-hydroxybenzoate | 0.2 |
| Hibiscin ® HP LS 9198 | 3.0 |
| TiO₂ | 0.5 |
| Citric acid | 0.1 |
| Ederline L | 3.0 |
| Calcium pantothenate | 0.048 |
| Sepigel ® 305 | 2.0 |
| Water | to 100 |

### Example 5

### Cream Formulation (Data in % by Weight)

| | |
|---|---|
| Lipoid S 75-3 | 1.50 |
| Baysilon ® M 350 | 1.00 |
| Cetiol ® J 600 DEO | 4.00 |
| Cetiol ® SB 45 | 3.00 |
| Stenol ® 1618 gesch. | 0.50 |
| Cutina ® MD-V | 1.00 |
| Floraesters 70 | 1.50 |
| Controx ® KS | 0.20 |
| Almond oil | 2.00 |
| Propyl p-hydroxybenzoate | 0.20 |
| Tego carbomer 140 2% strength | 20.00 |
| Talc Pharma G | 1.00 |
| Glycerol | 3.00 |
| Dipropylene glycol | 6.00 |
| Methyl p-hydroxybenzoate | 0.20 |
| Dry Flo PLUS | 1.00 |
| Retinyl palmitate | 0.10 |
| DSH-CN | 2.00 |
| Ederline L | 3.00 |
| NaOH 10% strength | 1.50 |
| Water | to 100 |

### Example 6

### Cream Formulation (Data in % by Weight)

| | |
|---|---|
| Baysilon ® M 350 | 1.00 |
| Cetiol ® OE | 5.00 |
| Cegesoft ® C 24 | 5.00 |
| Stenol ® 1618 gesch. | 2.00 |
| Cutina ® MD-V | 1.00 |
| Tego Care CG 90 | 1.00 |
| Propyl p-hydroxybenzoate | 0.10 |
| Glycerol | 5.00 |
| Sorbitol | 3.00 |
| Glucose | 1.00 |
| Methyl p-hydroxybenzoate | 0.10 |
| Ederline L | 5.00 |
| Water | to 100 |

### Example 7

### Cleansing Milk (Data in % by Weight)

| | |
|---|---|
| Tego carbomer 140 2% strength | 20.00 |
| Benecel | 0.30 |
| Paraffin oil | 20.00 |
| Stenol ® 1618 gesch. | 2.00 |
| Hostaphat KW 340 D | 3.00 |
| Eumulgin B1 | 1.50 |
| Tocopheryl acetate | 0.50 |
| Propyl p-hydroxybenzoate | 0.20 |
| Glycerol | 5.00 |
| Hexanediol | 3.00 |
| Methyl p-hydroxybenzoate | 0.20 |
| Ederline L | 5.00 |
| Glucono-Δ-lactone | 2.00 |
| Phenoxyethanol | 0.40 |
| Trilon M | 0.10 |
| Water | to 100 |

### Example 8

### Leave-on Hair Tonic (Data in % by Weight)

| | |
|---|---|
| MONOMULS ® 60-35 C | 1.24 |
| EMULGIN ® B 1 | 2.76 |
| Cetiol S | 9.00 |
| Cetiol OE | 9.00 |
| Dow Corning DC 345 ® | 2.00 |
| GLUADIN ® WQ | 2.85 |
| PLANTACARE ® 2000 UP | 2.00 |
| Ederline L | 5.00 |
| Water | to 100 |

List of the Ingredients Used

| Component | INCI name | Manufacturer |
|---|---|---|
| Baysilonöl ® M350 | Dimethicone | GE Bayer Silicones |
| Benecel MP 333 C | Hydroxypropyl | Hercules |
| | Methylcellulose | |
| Biophilic H | Hydrogenated Lecithin | Lucas Meyer |
| | Fatty Acids, Fatty | |
| | Alcohols | |
| Cegesoft C 24 | Ethylhexyl Palmitate | Cognis |
| Cetiol ® J 600 DEO | Oleyl Erucate | Cognis |
| Cetiol ® OE | Dicaprylyl Ether | Cognis |
| Cetiol ® S | Dioctylcyclohexane | Cognis |
| Cetiol ® SB 45 | Butyrospermum Parkii | Cognis |
| | (Linne) | |
| Controx ® KS: | Tocopherol, | Cognis |
| | Hydrogenated Palm | |
| | Glycerides Citrate | |
| Cutina ® GMS (C16-18 | Glyceryl Stearate | Cognis |
| fatty acid | | |
| mono-diglyceride | | |
| Cutina ® MDV (C16-18 | Glyceryl Stearate | Cognis |
| fatty acid | | |
| mono-diglyceride | | |
| Dow Corning DC 345 | Cyclomethicone | Dow Corning |
| Dry Flo Plus | Aluminium Starch | National Starch and |
| | Octenylsuccinate | Chemical Company |
| DSH-C-N | Dimethylsilanol | Exsymol |
| | Hyaluronate (aqueous | |
| | solution) | |
| Emuliance | Cetearyl Wheat Bran | Soliance |
| | Glycosides, Cetearyl | |
| | Alcohol | |
| Eumulgin B1 | Ceteareth-12 | Cognis |
| Eusolex ® 4360 | Benzophenone-3 | Merck |
| Eusolex ® 6300 | 4-Methylbenzylidene | Merck |
| | Camphor | |
| Floraesters 60 | Jojoba Esters | Flora Technologies |
| Floraesters 70 | Jojoba Esters | Flora Technologies |
| Generol ® R | Brassica Campestris | Cognis |
| | (Rapeseed) Sterols | |
| Gluadin ® WQ | Laurdimonium | Cognis |
| | Hydroxypropyl | |
| | Hydrolyzed Wheat | |
| | Protein (31%) | |
| Hibiscin ® HP-LS 919 | Water, Hibiscus | Laboratoires |
| | Esculentus Seed | Serobiologiques |
| | Extract, Phenoxyethane | |
| Hostaphat KW 340 D | C16-C18-fatty alcohol | Clariant |
| | 4-EO-orthophosphoric | |
| | acid mono-di-triester | |
| Lanette ® 22 | Behenyl Alcohol | Cognis |
| Lipoid S75-3 | Hydrogenated Lecithin | Lipoid GmbH |
| MONOMULS ® 60-35 | Hydrogenated Palm | Cognis |
| C | Glycerides | |
| Montanov ® 202 | Arachidyl Alcohol, | SEPPIC |
| | Behenyl Alcohol, | |
| | Arachidyl Glucoside | |
| Myritol ® 318 | Caprylic/Capric | Cognis |
| | Triglyceride | |
| Myritol ® 331 | Cocoglycerides | Cognis |
| Myritol ® PC | Propylene Glycol | Cognis |
| | Dicaprylate/Dicaprate | |
| Novata ® AB | Coconut Glycerides | Cognis |
| Parsol ® 1789 | Butyl Methoxydibenzoyl | Roche |
| | methane | |
| Performalene 400- | Polyethylene | New |
| Polyethylene | | Phase Technologies |
| PLANTACARE ® 2000 | Decyl Glucoside (about | Cognis |
| UP | 50%) | |
| Sepigel ® 305 | Polyacrylamide, C13-14 | SEPPIC |
| | Isoparaffin, Laureth-7 | |
| Stenol ® 16/18 | Cetearyl Alcohol | Cognis |
| Tego Care CG 90 | Cetearyl Glucoside, at | Goldschmidt |
| | least 90% active | |
| | substance | |
| Trilon ® A | Nitrilotriacetic acid | BASF |
| | Na | |
| Trilon ® M | Methylglycinediacetic | BASF |
| | acid trisodium salt | |
| Uvinul ® T 150 | Octyl Triazone | BASF |

1. A topical cosmetic or pharmaceutical composition comprising at least one apple seed extract and at least one active ingredient selected from the group consisting of:
organic, inorganic and modified inorganic light protection filters,
protein hydrolyzates and their derivatives,
mono-, oligo- and polysaccharides, and their derivatives, and
α-hydroxycarboxylic acids and α-ketocarboxylic acids, and their ester, lactone or salt forms;
in a suitable carrier.

2. A method of improving the mechanical stability of the skin, the skin appendages and the hair comprising adding apple seed extract to topical cosmetic or pharmaceutical compositions.

3. The method of claim 2 wherein the mechanical stability is caused by the increase in the number of keratinocytes in the skin and the skin appendages.

4. The method of claim 2 wherein the mechanical stability is caused by the increase in the keratinocyte differentiation in the skin and the skin appendages.

5. The method of claim 2 wherein the mechanical stability is caused by the increase in the intercellular cell adhesion in the skin and the skin appendages.

6. The method of claim 2 wherein the mechanical stability is caused by the increase in the adhesion between cells and the extracellular matrix in the skin and the skin appendages.

7. A method for stimulating the endogenous barrier function of the skin and the skin appendages comprising adding apple seed extracts to topical cosmetic or pharmaceutical compositions.

8. The method of claim 7 wherein the stimulation is caused by an increase in the lipid production in the epidermis.

9. The method of claim 7 wherein the stimulation is caused by an increase in the formation of intercellular communication pores in the skin and the skin appendages and by the improvement in the intercellular cell communication.

10. A method for increasing the expression:
of the keratin 5 (KRT5) having the Swiss-Prot number P13647,
of the keratin 14 (KRT14) having the Swiss-Prot number P02533,
of the keratin 10 (KRT10) having the Swiss-Prot number P13645,
of the keratin 1 (KRT1) having the Swiss-Prot number P04264,
of the gap junction protein beta 2 connexin 26 (CXB 2) having the Swiss-Prot number P29033,
of the gap junction protein alpha 1 connexin 43 having the Swiss-Prot number P17302,
of the hyaluronic acid receptor CD44 having the Swiss-Prot number P16070,
of the fatty acid synthase (FAS) having the enzyme classification EC 2.3.1.85 and the Swiss-Prot number P49327,
of the epidermal fatty acid binding protein FABE having the Swiss-Prot number Q01469,
of the protein PSMD2 having the Swiss-Prot number Q13200 and
of the DNA binding protein A (DBPA) having the Swiss-Prot number P16989,
in cells of the skin and the skin appendages comprising adding apple seed extracts to topical cosmetic or pharmaceutical compositions.

11. A method for inhibiting the expression of the bone proteoglycan II precursor (PGS2) having the Swiss-Prot number P07585 in cells of the skin and the skin appendages comprising adding apple seed extracts to topical cosmetic or pharmaceutical compositions.
